(19) United States
(12) Patent Application Publication
(10) Pub. No.: US 2008/0213910 A1
(43) Pub. Date: Sep. 4, 2008

(54) METHOD FOR BLOCKING NON-SPECIFIC PROTEIN BINDING ON A FUNCTIONALIZED SURFACE

(76) Inventor: Gangadhar JOGIKALMATH, Belmont, MA (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 11/925,593

(22) Filed: Oct. 26, 2007

Related U.S. Application Data
(60) Provisional application No. 60/863,584, filed on Oct. 31, 2006.

(51) Int. Cl. G01N 33/00; B01J 19/00

(52) U.S. Cl. 436/86; 422/68.1

(57) ABSTRACT

A method of blocking non-specific protein binding on surfaces, such as protein-coated biosensor surfaces.

This application claims priority to U.S. Provisional Application No. 60/863,584 filed Oct. 31, 2006, which is hereby incorporated by reference herein in its entirety, including the drawings.

### FIELD OF THE INVENTION

This invention relates to methods of blocking non-specific protein binding on a surface. This invention also relates to biosensor surfaces that have been blocked by monosaccharides, disaccharides (such as trehalose), polysaccharides (such as dextran), or oligosaccharides to prevent non-specific protein binding.

### BACKGROUND OF THE INVENTION

With the completion of the sequencing of the human genome, one of the next grand challenges of molecular biology will be to understand how the many protein targets encoded by DNA interact with other proteins, small molecule pharmaceutical candidates, and a large host of enzymes and inhibitors. See e.g., Pandey & Mann, “Proteomics to study genes and genomes,” Nature, 405, p. 837-846, 2000; Leigh Anderson et al., “Proteomics: applications in basic and applied biology,” Current Opinion in Biotechnology, 11, p. 408-412, 2000; Patterson, “Proteomics: the industrialization of protein chemistry,” Current Opinion in Biotechnology, 11, p. 413-418, 2000; MacBeath & Schreiber, “Printing Proteins as Microarrays for High-Throughput Function Determination,” Science, 289, p. 1760-1763, 2000; De Wildt et al., “Antibody arrays for high-throughput screening of antibody-antigen interactions,” Nature Biotechnology, 18, p. 989-994, 2000. To this end, tools that have the ability to simultaneously identify and/or quantify many different biomolecular interactions with high sensitivity will find application in pharmaceutical discovery, proteomics, and diagnostics. Further, for these tools to find widespread use, they must be simple to use, inexpensive to own and operate, and applicable to a wide range of analytes that can include, for example, polynucleotides, peptides, small proteins, antibodies, and even entire cells.

The immobilization of target molecules onto support surfaces has become an important aspect in the development of biological assays. Generally, biological assays are carried out on the surfaces of microwell plates, microscope slides, tubes, silicone wafers or membranes. The target molecules are covalently immobilized on the surface using coupling reactions between the functional groups on the surface and the functional groups of the molecules. One of popular surface functionalization techniques on glass surface is silanization using functional silanes. Silane, Silicones, and Metal-Organics, p. 88, published by Gelest Inc., Tullytown, Pa. (2000). GAPS II coated slides manufactured by Corning Inc. (Corning, N.Y.), Arryit™ SuperAmine slides supplied by TeleChem International, Inc (Sunnyvale, Calif.), SILANE-PREP™ amine-functionalized slides provided by Sigma Diagnostics (St Louis, Mo.) and others are examples of available biological assay surfaces in microscope slide format. The SuperAmine slide is claimed to provide 5×10¹² amine groups per mm². As another example, amide groups that have been derivatized amidine on a nylon support are used to immobilize DNA and RNA probes in hybridization assays to detect specific polynucleotide sequences. See U.S. Pat. No. 4,806,546. Products in formats of microwell plates and tubes, including NucleoLink™ and CovaLink™ provided by Nalge Nunc International (Rochester, N.Y.), are available only on polymeric support surfaces. The CovaLink™ products provide a secondary amine surface at approximately 10¹² groups per mm² of surface area. Secondary amines show a lower reactivity than primary amines in many conjugation reactions. See, Loudon, G. Marc, Organic Chemistry, 3d ed., The Benjamin/Cummings Publishing, Redwood City, Calif. (1995).

There are numerous known methods for chemically functionalizing the surfaces of materials, such as silicon, glass or gold, for example. Surface functionalization is of great interest, as it often leads to expanded applications for the surface, whereby enhanced binding and analysis of various molecules to the surface becomes possible, relative to a surface with a non-chemically functionalized surface. The type, quantity, and quality of a chemical functionalization coating on a surface determine the covalent strength and capacity of the surface to bind a particular analyte. It is highly desirable that the coating itself not be easily washed away or degraded after multiple uses.

Aldehyde-functional groups and amine-functional groups coated on a surface have been shown to provide a versatile platform for detecting biomolecules. These groups can capture biomolecules through physical attraction, such as electrostatic interaction, for example, or chemical binding. Such chemical binding can be achieved directly or indirectly (i.e. through a chemical linker). Many homobifunctional or heterobifunctional linkers are known in the field. A simple method for coating a surface with amine is to directly expose the cleaned surface to polylysine. An example is a glass slide surface used for microarray printing. An alternative to coating a surface with amines is to covalently attach amine-coating molecules to the surface, such as attaching silanes on glass or thiols on gold, both of which are well known.

Various aminoalkylsilane reagents have been used to coat silicon- or glass-based surfaces with amine groups. Processes used in coating such surfaces include the use of a variety of silane reagents, solvents, and different physical treatment procedures. Further, to test the presence of a chemical group on a surface, methods including radioactive, calorimetric, fluorescence, XPS, FTIR, AFM and other methods have been used. Sensitivity is an important issue when selecting the appropriate method for surface testing. Generally speaking, there is neither a standard industry procedure to chemically coat a biosensor sensor surface, nor a standardized testing method for detecting the presence or quantity of a particular chemical moiety on such a biosensor.

One method of coating a surface with aldehyde binding sites is functionalizing the surface with amine groups and adding an aldehyde solution comprising cyanoborohydride to the amine-functionalized surface. The resulting biosensors can be used for binding proteins and other amine-containing molecules. Some aldehyde-modified slides are also commercially available (e.g., CEL Associates and NoAb BioDiscoveries) for printing arrays.

Biosensors have been developed, for example, to detect a variety of biomolecular complexes including oligonucleotides, antibody-antigen interactions, hormone-receptor interactions, and enzyme-substrate interactions. In general, biosensors consist of two components: a highly specific recognition element and a transducer that converts the molecular recognition event into a quantifiable signal. Signal transduction has been accomplished by many methods, including fluorescence, interferometry (Jenison et al., “Interference-based detection of nucleic acid targets on optically coated silicon,” Nature Biotechnology, 19, p. 62-65; Lin et al., “A porous silicon-based optical interferometric biosensor,” Science, 278, p. 840-843, (1997)), and gravimetry (A. Cunningham, Bioanalytical Sensors, John Wiley & Sons (1998)).

Of the optically-based transduction methods, direct methods that do not require labeling of analytes with fluorescent compounds are of interest due to the relative assay simplicity and ability to study the interaction of small molecules and proteins that are not readily labeled. Direct optical methods include surface plasmon resonance (SPR) (Jordan & Corn, “Surface Plasmon Resonance Imaging Measurements of Electrostatic Biopolymer Adsorption onto Chemically Modified Gold Surfaces,” Anal. Chem., 69:1449-1456 (1997)), grating couplers (Morhard et al., “Immobilization of antibodies in micropatterns for cell detection by optical diffraction,” Sensors and Actuators B, 70, p. 232-242, (2000)), ellipsometry (Jin et al., “A biosensor concept based on imaging ellipsometry for visualization of biomolecular interactions,” Analytical Biochemistry, 232, p. 69-72, (1995)), evanescent wave devices (Huber et al., “Direct optical immunosensing (sensitivity and selectivity),” Sensors and Actuators B, 6, p. 122-126, (1992)), and reflectometry (Brecht & Gauglitz, “Optical probes and transducers,” Biosensors and Bioelectronics, 10, p. 923-936, (1995)). Theoretically predicted detection limits of these detection methods have been determined and experimentally confirmed to be feasible down to diagnostically relevant concentration ranges.

Aldehyde-functionalized surfaces have been used to immobilize or capture a target molecule on the surface of several device formats including microarray, micro-well plate, and well slide. After the target molecule has been immobilized on the surface, it can bind analyte molecules in an unknown sample by specific molecular interaction in order to analyze the sample. The unreacted aldehyde groups, however, remain reactive with the ability to attach the analyte molecules chemically onto the surface with the target molecule-analyte interactions, thereby resulting in binding that is non-specific. Many chemical and biological molecules and cells tend to adsorb to most surfaces through hydrophobic interaction and/or charge interaction, even without any target molecules, such as biological receptors, on the surface. This adsorption also causes unwanted non-specific binding. Non-specific binding reduces signal-to-noise ratios in biomolecules detection based on specific biomolecules interaction. The aldehyde density of biosensors, including the BIND™ sensors, has increased dramatically. As the aldehyde density increases, non-specific binding reaches a significant level, causing difficulty in determining whether detection signals are from analytes or from false readings. Therefore, the reduction of non-specific binding is of great interest. It is also of great interest to reduce non-specific binding to amine-functionalized surfaces.

As an example, a target molecule, such as streptavidin, can be immobilized on a biosensor surface. The biosensor can measure the binding of other proteins or small molecules, also known as ligands, to the target molecule by measuring the change in signal generated by such a binding event. This signal can be, for example, optical, electrical, or visual. However, the biosensor surface containing the target molecule needs to be blocked so that non-specific binding of proteins or small molecules to the surface is reduced and only the specific interactions with target molecules are allowed. An example of such a specific interaction is the interaction between streptavidin and biotin, the specific ligand for streptavidin. Blocking has typically been accomplished by using any of the available commercial blockers such as Superblock® Blocking Buffer, Sea Block Blocking buffer, Blockerit, Blocker Casin, Fish skin gelatin, and BSA, such as 1% BSA+TWEEN®. These blockers are amphipathic and can merely trade one unwanted attraction for another. Previous attempts to block streptavidin-coated biosensors, such as BIND™ sensor plates, resulted in the reduction of activity of the streptavidin due to the interference by such commercially available blockers, as measured in terms of reduced biotin binding to streptavidin. This interference could be due, in part, to the presence of biological materials, such as proteins (or peptides) present in the commercial blockers. Therefore, there remains a need in the art to address this issue.

### SUMMARY OF THE INVENTION

One embodiment of the invention provides a method for reducing non-specific binding on a surface, wherein the surface is aldehyde-functionalized, amine-functionalized, or a combination thereof. The method comprises treating the surface with sugar molecules, whereby non-specific binding on the surface is reduced. The sugar molecules can comprise disaccharides. The disaccharides can be trehalose molecules or comprise trehalose molecules. The sugar molecule can also be or comprise dextran sulfate. The surface can be a biosensor surface, such as a calorimetric resonant reflectance biosensor surface. The sugar molecules can comprise monosaccharides, disaccharides, polysaccharides, trisaccharides, tetrasaccharides, pentasaccharides, or a combination thereof. The surface can be an amine-functionalized surface. The sugar molecules can comprises lactose, glyceraldehydes, or a combination thereof. The sugar molecules comprise trehalose and lactose. The sugar molecules can comprise trehalose and glyceraldehyde.

Another embodiment of the invention provides a biosensor comprising a plurality of specific binding substances bound to surface-attached aldehyde groups and a plurality of sugar molecules bound to surface-attached aldehyde groups. The sugar molecules can be or comprise disaccharides. The disaccharides can be trehalose molecules. Alternatively, the sugar molecule can be or comprise dextran. The specific binding substances can be proteins. The proteins can comprise streptavidin.

Even another embodiment of the invention provides a package containing a biosensor with specific binding substances bound to surface-attached aldehyde groups and a storage solution comprising sugar molecules.

Therefore, the invention provides small molecules with high hydrophilic, non-charged characteristics that do not provide unwanted attractions like amphipathic blockers.

### BRIEF DESCRIPTION OF THE FIGURES

FIGS. 1A and 1B are schematic diagrams of various embodiments of an optical grating structure used for a calorimetric resonant reflectance biosensor. n_{substrate} represents substrate material. n₁ represents the refractive index of a cover layer. n₂ represents the refractive index of a one- or two-dimensional grating. n_{bio} represents the refractive index of one or more specific binding substances. t₁ represents the thickness of the cover layer. t₂ represents the thickness of the grating. t_{bio} represents the thickness of the layer of one or more specific binding substances.

FIG. 2 represents the shift, in nm, resulting from a 5% trehalose solution added to either streptavidin (SA)-immobilized wells, or control wells containing only the 5% trehalose solution. SA concentrations during immobilization were either 0.05 mg/ml or 0.2 mg/ml.

FIG. 3 represents the shift, in nm, resulting from a 5% trehalose solution added to either streptavidin (SA)-immobilized wells, followed by either 10% or 100% FBS. Results are compared to wells without trehalose. SA concentrations during immobilization were either 0.05 mg/ml or 0.2 mg/ml.

FIG. 4 represents the normalized peak wavelength value (PWV) resulting from warfarin binding to HSA after blocking with 5% trehalose.

FIG. 5 represents the normalized peak wavelength value (PWV) resulting from CBS binding to carbonic anhydrase after blocking with 5% trehalose.

### DETAILED DESCRIPTION OF THE INVENTION

Functionalized-coated surfaces of biosensors are useful for binding chemical or biological molecules such as proteins, peptides, polypeptides, nucleotides, polynucleotides, small molecules, small organic molecules, biotin, cells, fractionated cells, cells extracts, cell fractions, parts of cells and other chemical or biological molecules that are of interest in the areas of, for example, proteomics, genomics, pharmaceuticals, drug discovery, and diagnostic studies. For example, biosensors can be aldehyde-coated or amine-coated to bind chemical or biological molecules that are of interest.

As used herein, “aldehyde” refers to molecules having the formula —CHO and “amine” refers to both primary amines having the formula —NH₂ and secondary amines. Aldehydes and amines may be attached directly or through a linking molecule to a surface, such as the surface of a biosensor. An amine-coated surface or an amine-functionalized surface refers to a surface which provides amine groups available for chemical modification, such as the attachment of specific binding substances, either directly or indirectly. An aldehyde-coated surface or an aldehyde-functionalized surface refers to a surface which provides aldehyde groups available for chemical modification, such as the attachment of specific binding substances, either directly or indirectly. Indirect attachment refers to the attachment of chemical or biological molecules through a chemical linker as is well known in the art.

As used herein, “surface” refers to any surface that is capable of binding specific binding substances, either directly of indirectly. For examples, a surface can contain functional amine or functional aldehyde groups attached directly to the surface, or the functional groups can be attached to the surface by a linker molecule. A surface may or may not be functionalized. A surface can be, but is not limited to, plastic, glass, or gold. Such surfaces include, but are not limited to, sensor surfaces, such as biosensor surfaces.

Also as used herein, sugar molecules refer to monosaccharides, disaccharides, polysaccharides, and oligosaccharides. Disaccharides, polysaccharides, or oligocaccharides can be, but are not limited to trehalose, lactose, maltose, maltotriose, palatinose, lactulose, sucrose, dextran, raffinose, stachyose, verbascose. Trehalose is a disaccharide composed of two glucose molecules bound by an α-1,1 linkage. See Higashiyama, Pure Appl Chem., 74(7):1263-1269 (2002).

A biosensor grating can be coated with a material having a high refractive index, for example, tantalum oxide, or other suitable material, optionally followed by an overcoat of silicon oxide. The ability to produce a high-sensitivity biosensor in plastic over a large surface area enables incorporation of the biosensor into large area, disposable assay formats, such as microtiter plates and microarray slides. Preferably, a biosensor can be incorporated into the bottom of a bottomless microtiter plate, microarrary, or a microfluidic device, and the biosensor plate can be used to perform, for example, multiple protein-protein or target molecule-ligand binding assays, in parallel. The bottomless microtiter plate can have, for example, 6, 8, 12, 24, 48, 96, 384, 1536, or 3456 wells. The detection sensitivity of a plastic-substrate biosensor is found to be superior or equivalent to previously reported glass-substrate biosensors. For example, plastic-based biosensors can be mass-produced; biosensor arrays, such as 96-well or 384-well, for example, can be up-scaled and mass-produced.

Plastic-based biosensors, or plastic biosensors, refer to those biosensors that contain a plastic grating or sensor surface, a plastic support for the grating, also referred to as a substrate, and/or other plastic components. Such biosensors can be susceptible to degradation as the result of reaction conditions used to functionalize the surfaces of the biosensors. Plastics having optical qualities are preferred. The plastic can be clear and transparent without any particulate and can be capable of providing a smooth, flat finish. As an example, a biosensor can include a polyester substrate that supports an acrylic polymer-grating layer. As a further example, a biosensor can include a polycarbonate substrate that supports an epoxy grating layer. Other non-limiting examples of plastics include polyesters and polyurethanes. However, any plastic that provides optical qualities for use in a biosensor may be used. In another example, the grating surface is plastic, such that the plastic serves as both the substrate and the grating. Such biosensors have been susceptible to degradation as the result of reaction conditions that are typically used in the art to functionalize the surfaces of such biosensors. There are, however, functionalization methods that do not cause degradation of plastic-based biosensors. See, e.g., U.S. patent application Ser. No. 10/983,511, filed Nov. 11, 2004, incorporated by reference in its entirety. One skilled in the art will recognize that the methods of the invention can also be used with glass-based biosensors.

As an example, the biosensor can be a BIND™ sensor plate. The BIND™ system allows label-free detection of chemical and biological molecular interactions. The BIND™ system can include a BIND™ reader and 96- or 384-well microplate biosensor. The BIND™ system uses an optical effect to provide sensitive measurement of binding on the biosensor surface. The biosensor can be a nonstructured optical grating, which is incorporated into microwell plates in industry standard formats. The BIND™ system allows, among other things, measurement of chemical and biological molecular interactions using proteins, peptides, and cells. The BIND™ system can be used for affinity ranking with antigen and functional screening with cells, peptide epitope mapping and immunogenictiy screening.

An aldehyde-functionalized surface or amine-functionalized surface refers to a surface having a coating through which specific binding substances may be attached. For example, an aldehyde-functionalized surface can refer to, but is not limited to, a grating surface of a biosensor having a coating of a high refractive index material through which specific binding substances can be attached. Such high refractive index materials include, for example, silicon nitride, zinc sulfide, titanium dioxide or tantalum oxide.

Optionally, a silicon oxide layer can be coated on the high refractive index material prior to surface functionalization. Either the high refractive index material or the silicon oxide can be functionalized with aldehyde-functional groups or amine-functional groups for attachment of chemical and biological molecules. The reagents used to aldehyde functionalize or amine functionalize the grating surface coated with the high refractive index material are preferably compatible with the grating material and the substrate material, whether they are plastic or epoxy. While the grating is coated with the high refractive index material, which provides some protection of the grating material from the reagents used to aldehyde functionalize or amine functionalize the surface, the opposite side of the grating may still be exposed during the functionalization process. Likewise, when the grating is bound to a substrate, the opposite side of the substrate may be exposed to the functionalization reagents. Also, imperfections in the coating of the high refractive index material on the grating surface may result in areas of the upper side of the grating surface exposed. Thus, the materials of the various layers and the adhesion between layers should remain intact during functionalization and any subsequent assay procedures.

An aldehyde-functionalized surface or an amine-functionalized surface of a biosensor refers to plastic-based biosensors, as well as biosensors that are not plastic based. For example, a biosensor includes a titanium oxide-coated sensor, or additional sensors with high refractive index, low index of absorption coating or covering for the top layer and for the base material construction. In addition, silicon dioxide, in all of its various physical forms, or other material with low index of absorption and low refractive index, are contemplated. These biosensors are meant to be exemplary, and are not limiting of biosensors that have an aldehyde-functionalized surface or an amine-functionalized surface.

### Subwavelength Structured Surface (SWS) Biosensor

In one embodiment of the invention, a subwavelength structured surface (SWS) is used to create a sharp optical resonant reflection at a particular wavelength that can be used to track with high sensitivity the interaction of chemical or biological materials, such as specific binding substances or binding partners or both. A calorimetric resonant reflectance biosensor surface acts as a surface-binding platform for specific binding substances.

Subwavelength structured surfaces are an unconventional type of diffractive optic that can mimic the effect of thin-film coatings. (Peng & Morris, “Resonant scattering from two-dimensional gratings,” J. Opt. Soc. Am. A, Vol. 13, No. 5, p. 993, May; Magnusson, & Wang, “New principle for optical filters,” Appl. Phys. Lett., 61, No. 9, p. 1022, August, 1992; Peng & Morris, “Experimental demonstration of resonant anomalies in diffraction from two-dimensional gratings,” Optics Letters, Vol. 21, No. 8, p. 549, April, 1996). A SWS structure contains a surface-relief, one-dimensional or two-dimensional grating in which the grating period is small compared to the wavelength of incident light so that no diffractive orders other than the reflected and transmitted zeroth orders are allowed to propagate. See U.S. patent application Ser. Nos. 10/059,060 and 10/058,626, incorporated by reference in their entirety. A SWS surface narrowband filter can comprise a one-dimensional or two-dimensional grating sandwiched between a substrate layer and a cover layer that fills the grating grooves. Optionally, a cover layer is not used. When the effective index of refraction of the grating region is greater than the substrate or the cover layer, a guided mode resonant effect occurs. When a filter is designed properly, the one-dimensional or two-dimensional grating structure selectively couples light at a narrow band of wavelengths. The light undergoes scattering, and couples with the forward- and backward-propagating zeroth-order light. The guided mode resonant effect occurs over a highly localized region of approximately 3 microns from the point that any photon enters the structure. Because propagation of guided modes in the lateral direction are not supported, a waveguide is not created.

The reflected or transmitted color of this structure can be modulated by the addition of molecules such as specific binding substances or binding partners or both to the upper surface of the cover layer or the one-dimensional or two-dimensional grating surface. The added molecules increase the optical path length of incident radiation through the structure, and thus modify the wavelength at which maximum reflectance or transmittance will occur.

In one embodiment, a biosensor, when illuminated with white light, is designed to reflect only a single wavelength. When specific binding substances or target molecules, such as chemical and biological molecules, are attached to the surface of the biosensor, the reflected wavelength (color) is shifted due to the change of the optical path of light that is coupled into the grating. By linking specific binding substances to a biosensor surface, complementary binding partner molecules can be detected without the use of any kind of fluorescent probe or particle label. The detection technique is capable of resolving changes of, for example, 0.1 nm thickness of protein binding, and can be performed with the biosensor surface either immersed in fluid or dried.

A detection system consists of, for example, a light source that illuminates a small spot of a biosensor at normal incidence through, for example, a fiber optic probe, and a spectrometer that collects the reflected light through, for example, a second fiber optic probe also at normal incidence. Because no physical contact occurs between the excitation/detection system and the biosensor surface, no special coupling prisms are required and the biosensor can be easily adapted to any commonly used assay platform including, for example, microtiter plates and microarray slides. A single spectrometer reading can be performed in several milliseconds, thus it is possible to quickly measure a large number of molecular interactions taking place in parallel upon a biosensor surface, and to monitor reaction kinetics in real time.

This technology is useful in applications where large numbers of biomolecular interactions are measured in parallel, particularly when molecular labels would alter or inhibit the functionality of the molecules under study. High-throughput screening of pharmaceutical compound libraries with protein targets, and microarray screening of protein-protein interactions for proteomics are examples of applications that require the sensitivity and throughput afforded by the compositions and methods of the invention.

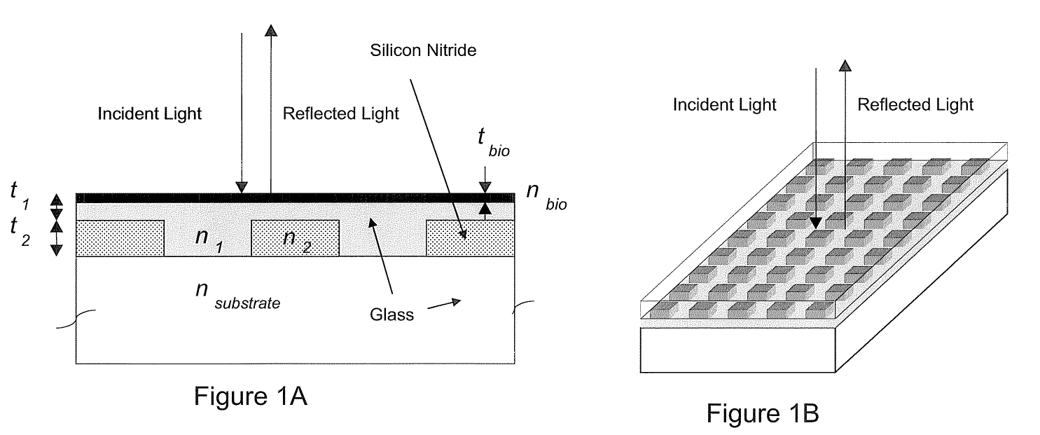

A schematic diagram of an example of a SWS structure is shown in FIG. 1. In FIG. 1, n_{substrate} represents a substrate material. n₁ represents the refractive index of an optional cover layer. n₂ represents the refractive index of a one- or two-dimensional grating. N_{bio} represents the refractive index of one or more specific binding substances. t₁ represents the thickness of the cover layer above the one- or two-dimensional grating structure. t₂ represents the thickness of the grating. t_{bio} represents the thickness of the layer of one or more specific binding substances. In one embodiment, n₂>n₁. (see FIG. 1). Layer thicknesses (i.e. cover layer, one or more specific binding substances, or a grating) are selected to achieve resonant wavelength sensitivity to additional molecules on the top surface. The grating period is selected to achieve resonance at a desired wavelength. The structures can be fabricated from glass and silicon nitride dielectric materials. Alternatively, structures may be formed from embossed plastic with an appropriate dielectric cover layer.

One embodiment of the invention provides a SWS biosensor. A SWS biosensor comprises a one-dimensional or two-dimensional grating, a substrate layer that supports the grating, and one or more specific binding substances immobilized on the surface of the grating opposite of the substrate layer.

A one-dimensional or two-dimensional grating can be comprised of a material, including, for example, zinc sulfide, titanium dioxide, tantalum oxide, and silicon nitride. A cross-sectional profile of the grating can comprise any periodically repeating function, for example, a “square-wave.” A grating can be comprised of a repeating pattern of shapes selected from the group consisting of continuous parallel lines squares, circles, ellipses, triangles, trapezoids, sinusoidal waves, ovals, rectangles, and hexagons. A sinusoidal cross-sectional profile is preferable for manufacturing applications that require embossing of a grating shape into a soft material such as plastic, or replicating a grating surface into a material such as epoxy. In one embodiment of the invention, the depth of the grating is about 0.01 micron to about 1 micron and the period of the grating is about 0.01 micron to about 1 micron.

A SWS biosensor can also comprise a one-dimensional linear grating surface structure, i.e., a series of parallel lines or grooves. A one-dimensional linear grating is sufficient for producing the guided mode resonant filter effect. While a two-dimensional grating has features in two lateral directions across the plane of the sensor surface that are both subwavelength, the cross-section of a one-dimensional grating is only subwavelength in one lateral direction, while the long dimension can be greater than wavelength of the resonant grating effect. A one-dimensional grating biosensor can comprise a high refractive index material that is coated as a thin film over a layer of lower refractive index material with the surface structure of a one-dimensional grating. Alternatively, a one dimensional grating biosensor can comprise a low refractive index material substrate, upon which a high refractive index thin film material has been patterned into the surface structure of a one-dimensional grating. The low refractive index material can be glass, plastic, polymer, or cured epoxy. The high refractive index material must have a refractive index that is greater than the low refractive index material. The high refractive index material can be zinc sulfide silicon nitride, tantalum oxide, titanium dioxide, or indium tin oxide, for example.

A SWS structure can be used as a microarray platform by, for example, building a grating surface that is the same size as a standard microscope slide and placing microdroplets of high affinity chemical receptor reagents onto an x-y grid of locations on the grating surface. Alternatively, the SWS structure can be built to be the same size as a standard microtiter plate, and incorporated into the bottom surface of the entire plate. When the chemically functionalized surface, for example the microarray/microtiter plate, is exposed to molecules, such as an analytes, the molecules will be preferentially attracted to locations that have high affinity. As a result, some surface locations gather additional material, and other surface locations do not. The surface locations that attract additional material can be determined by measuring the shift in resonant wavelength within each individual surface location, such as each individual microarry/microtiter surface location. Thus, for example, the amount of bound molecules, such as analytes, in the sample and the chemical affinity between receptor reagents and the molecules can be determined by measuring the extent of the shift of the resonant wavelength.

In one embodiment of the invention, an interaction of a first molecule with a second test molecule can be detected. A SWS biosensor as described above is used. Therefore, the biosensor comprises a one- or two-dimensional grating, a substrate layer that supports the one- or two-dimensional grating, and optionally, a cover layer. As described above, when the biosensor is illuminated a resonant grating effect is produced on the reflected radiation spectrum, and the depth and period of the grating are less than the wavelength of the resonant grating effect.

To detect an interaction of a first molecule with a second test molecule, a mixture of the first and second molecules is applied to a distinct location on a biosensor. A distinct location can be one spot or well on a biosensor or can be a large area on a biosensor. A mixture of the first molecule with a third control molecule is also applied to a distinct location on a biosensor. The biosensor can be the same biosensor as described above, or can be a second biosensor. If the biosensor is the same biosensor, a second distinct location can be used for the mixture of the first molecule and the third control molecule. Alternatively, the same distinct biosensor location can be used after the first and second molecules are washed from the biosensor. The third control molecule does not interact with the first molecule and is about the same size as the first molecule. A shift in the reflected wavelength of light from the distinct locations of the biosensor or biosensors is measured. If the shift in the reflected wavelength of light from the distinct location having the first molecule and the second test molecule is greater than the shift in the reflected wavelength from the distinct location having the first molecule and the third control molecule, then the first molecule and the second test molecule interact. Interaction can be, for example, hybridization of nucleic acid molecules, specific binding of an antibody or antibody fragment to an antigen, and binding of polypeptides. A first molecule, second test molecule, or third control molecule can be, for example, a nucleic acid, polypeptide, antigen, polyclonal antibody, monoclonal antibody, single chain antibody (scFv), F(ab) fragment, F(ab′)₂ fragment, Fv fragment, small organic molecule, cell, virus, and bacteria.

### Amine-Functionalized Biosensors

After a layer of high refractive index material, such as silicon nitride, is coated on a surface, such as a plastic surface, the device is prepared for use as a sensor by the attachment of amine-functional groups on the surface of the high refractive index material. Plastic-based biosensors can be degraded (i.e. structure or composition change on the sensor) during the chemical modification that provides amine functional groups on its surface. To avoid such degradation, the process for amine surface functionalization of a biosensor can use reagents that are compatible with the plastic of the biosensor. After a high refractive index material has been deposited on the grating surface of the plastic biosensor, the sensor may be stored or may be used directly for functionalization. The sensor may be subjected to a cleaning step using wet (e.g., cleaning using a liquid, such as solvent) or dry (e.g., UV ozone or plasma) methods prior to the amine functionalization procedure. In one embodiment, an amine functionalization procedure includes (a) exposing a plastic calorimetric resonant biosensor to an alcoholic silane solution, and then (b) rinsing the exposed plastic calorimetric resonant biosensor with an alcohol. When the biosensor is dried, the grating surface contains amine functional groups, i.e., —NH₂ groups.

In one embodiment, a silane solution includes a 3-aminopropyltriethoxysilane and an alcohol, such as ethanol or other suitable low molecular weight alcohol. Likewise any suitable low molecular weight alcohol may be used to rinse the biosensor. An example of coating a plastic biosensor with amine is first exposing the sensor to a solution containing 3-aminopropyltriethoxysilane and ethanol, then briefly rinsing the sensor in ethanol, and finally drying the sensor. The concentration of the 3-aminopropylsilane in ethanol may be adjusted such that the concentration of the 3-aminopropylsilane is from about 1% to about 15% in ethanol. In addition, the ethanol may be about 90%-100% (volume/volume, adjusted with water). The drying step may be done in an oven at about, 70° C. for 10 min, for example. The drying may be performed at higher temperatures, provided the temperature is selected such that sensor degradation does not occur.

Numerous suitable solvents, concentrations, reaction times, and curing/incubation times may be utilized. Variations include the type of surface, the silane reagent (other silane such as 3-aminopropyltrimethoxysilane, etc.), the silane concentration, the coating solvent or a combination of solvents (e.g. ethanol and water), the coating reaction time, the rinse solvent or a combination of solvents (e.g. ethanol and water), the curing time, and the curing temperature.

### Surface Treatment

In one embodiment of the invention, a sensor surface can be modified by chemical treatment. For example, the surface can be treated with a solution by immersing the surface in the solution. Alternatively, gas-phase treatment, including chemical vapor or atomization deposition can also be used for a coating of the surface. Gas-phase treatment can be used to ensure a conformal coating of the geometrically non-flat surface. Such a coating can be used in a step of silanizing a surface, or for the addition of other organic materials to a surface. Other methods by which a surface can be treated will be recognized by those skilled in the art.

Treatment by plasma can be commonly used prior to a gas-phase coating processes. A plasma treatment can remove most contamination on the surface and activate some of the surfaces to improve the adhesion of the subsequent gas-phase coating process.

A gas-phase coating process can be used to add chemical functionality and minimize adsorbed moisture, organic contaminants, and low molecular weight material, on the surface of polymer films. A gas-phase coating has advantages including, but not limited to, the uniform treatment of surfaces, no backside treatment when polymer films are treated, no pin-holes when treating porous materials. Such coating services useful in this invention include but are not limited services provided by Sigma Technologies (Tucson, Ariz.), 4th State (Belmont, Calif.), Yield Engineering (San Jose, Calif.), Erie Scientific (Portsmouth, N.H.), and AST Products (advanced surface technologies) (Billerica, Mass.).

### Acoustic Biosensors

In another embodiment of the invention, an acoustic biosensor is used. Acoustic biosensors measures the binding of a molecule, such as an analyte, to a chemical or biological molecule, or target molecule, that is covalently attached to the surface by detecting a change in the resonant oscillating frequency on the biosensor surface caused by a change in deposited mass as a result of the binding of the molecule and/or analyte. The resonant oscillating frequency can be measured, for example, by using piezoresistive devices, mechanical vibrators, such as micromachined cantilevers, membranes, or tuning forks, or surface acoustic wave oscillators.

### Electronic Biosensors

In another embodiment of the invention, an electronic biosensor is used. Electronic biosensors measures the binding of a molecule, such as an analyte, to a chemical or biological target molecule that is covalently attached to the surface by detecting a change of resistively, for example DC or AC, low or high frequency, capacitance, or inductance on the biosensor surface caused by a change in deposited mass as a result of the binding of the molecule and/or analyte.

### Specific Binding Substances and Binding Partners

One or more specific binding substances or target molecules can be immobilized on a surface, such as a sensor surface, by for example, physical adsorption or by chemical binding. A specific binding substance can, e.g., specifically bind to a binding partner that is added to the surface of the sensor. A specific binding substance specifically binds to its binding partner, but does not substantially bind other binding partners added to the surface of a biosensor. For example, where the specific binding substance is an antibody and its binding partner is a particular antigen, the antibody specifically binds to the particular antigen, but does not substantially bind other antigens. A specific binding substance can be, for example, a nucleic acid, peptide, polypeptide, protein, antigen, polyclonal antibody, monoclonal antibody, single chain antibody (scFv), F(ab) fragment, F(ab′)₂ fragment, Fv fragment, small molecule, small organic molecule, biotin, cell, cell extract, parts of cells, virus, bacteria, polymer, peptide solutions, single- or double-stranded DNA solutions, RNA solutions, solutions containing compounds from a combinatorial chemical library, or biological sample. A biological sample can be for example, blood, plasma, serum, gastrointestinal secretions, homogenates of tissues or tumors, synovial fluid, feces, saliva, sputum, cyst fluid, amniotic fluid, cerebrospinal fluid, peritoneal fluid, lung lavage fluid, semen, lymphatic fluid, tears, or prostatitc fluid.

Preferably, one or more specific binding substances are arranged in a microarray of distinct locations on a biosensor. A microarray of specific binding substances comprises one or more specific binding substances on a surface of a biosensor of the invention such that a surface contains many distinct locations, each with a different specific binding substance or with a different amount of a specific binding substance. For example, an array can comprise 1, 10, 100, 1,000, 10,000, or 100,000 distinct locations. Such a biosensor surface is called a microarray because one or more specific binding substances are typically laid out in a regular grid pattern in x-y coordinates. However, a microarray of the invention can comprise one or more specific binding substance layed out in any type of regular or irregular pattern. For example, distinct locations can define a microarray of spots of one or more specific binding substances. A microarray spot can be about 50 to about 500 microns in diameter. A microarray spot can also be about 150 to about 200 microns in diameter. One or more specific binding substances can be bound to their specific binding partners.

A microarray on a biosensor of the invention can be created by placing microdroplets of one or more specific binding substances onto, for example, an x-y grid of locations on a one- or two-dimensional grating or cover layer surface. When the biosensor is exposed to a test sample comprising one or more binding partners, the binding partners will be preferentially attracted to distinct locations on the microarray that comprise specific binding substances that have high affinity for the binding partners. Some of the distinct locations will gather binding partners onto their surface, while other locations will not.

One example of a microarray of the invention is a nucleic acid microarray, in which each distinct location within the array contains a different nucleic acid molecule. In this embodiment, the spots within the nucleic acid microarray detect complementary chemical binding with an opposing strand of a nucleic acid in a test sample.

While microtiter plates are the most common format used for biochemical assays, microarrays are increasingly seen as a means for maximizing the number of biochemical interactions that can be measured at one time while minimizing the volume of precious reagents. By application of specific binding substances with a microarray spotter onto a biosensor of the invention, specific binding substance densities of 10,000 specific binding substances/in² can be obtained. By focusing an illumination beam to interrogate a single microarray location, a biosensor can be used as a label-free microarray readout system.

### Immobilization of One or More Specific Binding Substances

Immobilization of one or more binding substances onto a biosensor is performed so that a specific binding substance will not be washed away by rinsing procedures, and so that its binding to binding partners in a test sample is unimpeded by the biosensor surface. Several different types of surface chemistry strategies have been implemented for covalent attachment of specific binding substances to, for example, glass for use in various types of microarrays and biosensors. These same methods can be readily adapted to a biosensor of the invention. Surface preparation of a biosensor so that it contains the correct functional groups for binding one or more specific binding substances is an integral part of the biosensor manufacturing process.

As used herein, the terms “target molecule” or “chemical or biological molecules” or “specific binding substances” refer to any specific binding substances that can be attached to the functionalized surface. Chemical or biological molecules can be selected from the group consisting of, e.g., proteins, peptides, polypeptides, nucleotides, polynucleotides, small molecules, biotin, cells, fractionated cells, cells extracts, cell fractions, and parts of cells.

As used herein, the terms protein, peptide and polypeptide refer to a polymer of amino acid residues. The terms also apply to amino acid polymers in which one or more amino acids are chemical analogues of corresponding naturally-occurring amino acids, including amino acids which are modified by post-translational processes (e.g., glycosylation and phosphorylation). The term “protein,” as used herein, means any protein, including, but not limited to peptides, enzymes, glycoproteins, hormones, receptors, antigens, antibodies, growth factors, etc., without limitation.

The term “polypeptide” refers to a polymer of amino acids without regard to the length of the polymer; thus, peptides, oligopeptides, and proteins are included within the definition of polypeptide. This term refers to both naturally occurring polypeptides and synthetic polypeptides. This term can include chemical or post-expression modifications of the polypeptide. Therefore, for example, modifications to polypeptides which include the covalent attachment of glycosyl groups, acetyl groups, phosphate groups, lipid groups and the like are expressly encompassed by the term polypeptide. A chemically modified polypeptide includes polypeptides where an identification or capture tag has been incorporated into the polypeptide. The natural or other chemical modifications, such as those listed in example above, can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, hydrogenation, iodination, methylation, myristoylation, oxidation, pegylation, proteolytic processing, phosphorylation, prenylation, racemization, selenylation, sulfation, transfer-RNA mediated addition of amino acids to proteins such as arginylation, and ubiquitination. (See, e.g., Proteins—Structure and Molecular Properties, 2nd Ed., T. E. Creighton, W. H. Freeman and Company, New York (1993); Posttranslational Covalent Modification of Proteins, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992)). Also included within the definition are polypeptides which contain one or more analogs of an amino acid (including, for example, non-naturally occurring amino acids, amino acids that only occur naturally in an unrelated biological system, modified amino acids from mammalian systems etc.), polypeptides with substituted linkages, as well as other modifications known in the art, both naturally occurring and non-naturally occurring. The polypeptide may be naturally occurring or synthetic

As used herein, “small molecule” refers to molecules that are less than around 2,500 Daltons. These molecules include, for example, small organic molecules, such as biotin, but also include small peptides, such as peptidomimetics, as well as nucleotides, such as those commonly found in antiviral screening libraries. Small molecules can refer to both synthetic molecules, which can be diversity oriented and smaller in size, and natural compounds, which tend to have larger molecular weights. Small molecules also include orally acting drugs, which have an upper size range of about 500 Daltons. See Lipinski, C.A. “Drug-like Properties and the Causes of Poor Solubility and Poor Permeability,” J. Pharm. And Tox. Methods. 44:235 (2000) at 236 (“Lipinski”).

One or more specific binding substances can be attached to a biosensor surface by physical adsorption (i.e., without the use of chemical linkers) or by chemical binding (i.e., with the use of chemical linkers). Chemical binding can generate stronger attachment of specific binding substances on a biosensor surface and provide defined orientation and conformation of the surface-bound molecules.

Examples of types of chemical binding include, for example, amine functionalization, aldehyde functionalization, carboxyl functionalization, and biotin, glutathione-S-transferase (GST), and nickel activation. These surfaces can be used to attach specific binding substances directly to a biosensor surface or through the use of several different types of chemical linkers, as shown in Table 1. See also, Hermanson, Bioconjugate Techniques, Academic Press, NY, 1996.

TABLE 1

| Sensor | | Targeted Groups on |
|---|---|---|
| Surface | | Specific Binding |
| Group | Chemical Linkers | Substances |
| Amine | Sulfosuccinimidyl-6-(biotinamido)hexanoate | Streptavidin or |
| | (sulfo-NHS-LC-biotin) | avidin |
| | N,N′-disuccinimidyl carbonate | Amine |
| | (DSC, non-cleavable linker) | |
| | Dimethyl 3,3′-dithiobispropionimidate | Amine |
| | (DTBP, cleavable linker) | |
| | 1-Ethyl-3-(3-Dimethylaminopropyl)carbodiimide (EDC)/ | carboxyl |
| | N-Hydroxysulfosuccinimide (NHS) | |
| | Sulfo-succinimidyl 6-[a-methyl-a-(2-pyridyl-dithio) | sulfhydryl |
| | toluamido]hexanoate (Sulfo-LC-SMPT, cleavable linker), | |
| | Sulfo-succinimidyl 4-(N-maleimidomethyl) cyclohexane- | sulfhydryl |
| | 1-carboxylate (Sulfo-SMCC, non-cleavable linker) | |
| Aldehyde | | Amine |
| Carboxyl | | Amine |
| Nickel (II) | | His-tagged |
| | | biomolecules |
| Biotin | | Streptavidin or |
| | | avidin |
| Glutathione | | GST-tagged |
| | | biomolecules |

While an amine functionalized surface can be used to attach several types of linker molecules, an aldehyde functionalized surface can be used to bind proteins directly, without an additional linker. For example, an aldehyde functional coating on a surface is less than about 50 Angstroms thick. Also, the surface can be flat or not flat. A “not flat” surface can be, for example, a surface comprising a grating, as described herein. A “flat” surface can be, for example, a surface comprising a grating with an overcoat, such as silicon oxide or spin-on-glass (SOG), as described, for example, in U.S. patent application Ser. No. 09/930,352, filed Aug. 15, 2001 and U.S. patent application Ser. No. 10/059,060, filed Jan. 29, 2002 (which are incorporated by reference). A nickel surface can be used to bind molecules that have an incorporated histidine (“his”) tag. Detection of “his-tagged” molecules with a nickel-activated surface is well known in the art (Whitesides, Anal. Chem. 68, 490, (1996)).

Immobilization of specific binding substances to the surface of the plastic sensor, which can be an oxide, for example, can be performed essentially as described for immobilization to glass. However, the wash and coating treatment steps that would damage the material to which the specific binding substances are immobilized should be eliminated.

For the detection of binding partners at concentrations less than about 0.1 ng/ml, it is preferable to amplify and transduce binding partners bound to a biosensor into an additional layer on the biosensor surface. The increased mass deposited on the biosensor can be easily detected as a consequence of increased optical path length. By incorporating greater mass onto a biosensor surface, the optical density of binding partners on the surface is also increased, thus rendering a greater resonant wavelength shift than would occur without the added mass. The addition of mass can be accomplished, for example, enzymatically, through a “sandwich” assay, or by direct application of mass to the biosensor surface in the form of appropriately conjugated beads or polymers of various size and composition. This principle has been exploited for other types of optical biosensors to demonstrate sensitivity increases over 1500× beyond sensitivity limits achieved without mass amplification. See, e.g., Jenison et al., “Interference-based detection of nucleic acid targets on optically coated silicon,” Nature Biotechnology, 19: 62-65, 2001.

As an example, an amine functionalized sensor surface can have a specific binding substance comprising a single-strand DNA captured probe immobilized on the surface. The capture probe interacts selectively with its complementary binding partner. The binding partner, in turn, can be designed to include a sequence or tag that will bind a “detector” molecule. A detector molecule can contain, for example, a linker to horseradish peroxidase (HRP) that, when exposed to the correct enzyme, will selectively deposit additional material on the biosensor only where the detector molecule is present. Such a procedure can add, for example, 300 angstroms of detectable biomaterial to the biosensor within a few minutes.

A “sandwich” approach can also be used to enhance detection sensitivity. In this approach, a large molecular weight molecule can be used to amplify the presence of a low molecular weight molecule. For example, a binding partner with a molecular weight of, for example, about 0.1 kDa to about 20 kDa, can be tagged with, for example, succinimidyl-6-[a-methyl-a-(2-pyridyl-dithio)toluamido]hexanoate (SMPT), or dimethylpimelimidate (DMP), histidine, or a biotin molecule. Where the tag is biotin, the biotin molecule will binds strongly with streptavidin, which has a molecular weight of 60 kDa. Because the biotin/streptavidin interaction is highly specific, the streptavidin amplifies the signal that would be produced only by the small binding partner by a factor of 60.

Detection sensitivity can be further enhanced through the use of chemically derivatized small particles. “Nanoparticles” made of colloidal gold, various plastics, or glass with diameters of about 3-300 nm can be coated with molecular species that will enable them to covalently bind selectively to a binding partner. For example, nanoparticles that are covalently coated with streptavidin can be used to enhance the visibility of biotin-tagged binding partners on the biosensor surface. While a streptavidin molecule itself has a molecular weight of 60 kDa, the derivatized bead can have a molecular weight of any size, including, for example, 60 KDa. Binding of a large bead will result in a large change in the optical density upon the biosensor surface, and an easily measurable signal. This method can result in an approximately 1000× enhancement in sensitivity resolution.

### Methods of Using Sensors

Sensors of the invention can be used to study one or a number of specific binding substance/binding partner interactions in parallel. Binding of one or more specific binding substances to their respective binding partners can be detected, without the use of labels, by applying one or more binding partners to a biosensor that has one or more specific binding substances immobilized on its surfaces. For example, an SWS biosensor is illuminated with light and a maxima in reflected wavelength, or a minima in transmitted wavelength of light is detected from the biosensor. If one or more specific binding substances have bound to their respective binding partners, then the reflected wavelength of light is shifted as compared to a situation where one or more specific binding substances have not bound to their respective binding partners. Where a SWS biosensor is coated with an array of distinct locations containing the one or more specific binding substances, then a maxima in reflected wavelength or minima in transmitted wavelength of light is detected from each distinct location of the biosensor.

A variety of specific binding substances, for example, antibodies, can be immobilized in an array format onto a biosensor. The biosensor is then contacted with a test sample of interest comprising binding partners, such as proteins. Only the proteins that specifically bind to the antibodies immobilized on the biosensor remain bound to the biosensor. Such an approach is essentially a large-scale version of an enzyme-linked immunosorbent assay; however, the use of an enzyme or fluorescent label is not required.

The activity of an enzyme can be detected by applying one or more enzymes to a biosensor to which one or more specific binding substances have been immobilized. For example, the biosensor is washed and illuminated with light. The reflected wavelength of light is detected from the biosensor. Where the one or more enzymes have altered the one or more specific binding substances of the biosensor by enzymatic activity, the reflected wavelength of light is shifted.

Additionally, a test sample, for example, cell lysates containing binding partners can be applied to a biosensor, followed by washing to remove unbound material. The binding partners that bind to a biosensor can be eluted from the biosensor and identified by, for example, mass spectrometry. Optionally, a phage DNA display library can be applied to a biosensor of the invention followed by washing to remove unbound material. Individual phage particles bound to the biosensor can be isolated and the inserts in these phage particles can then be sequenced to determine the identity of the binding partner.

For the above applications, and in particular proteomics applications, the ability to selectively bind material, such as binding partners from a test sample onto a biosensor of the invention, followed by the ability to selectively remove bound material from a distinct location of the biosensor for further analysis is advantageous. Biosensors of the invention are also capable of detecting and quantifying the amount of a binding partner from a sample that is bound to a biosensor array distinct location by measuring the shift in reflected wavelength of light. For example, the wavelength shift at one distinct biosensor location can be compared to positive and negative controls at other distinct biosensor locations to determine the amount of a binding partner that is bound to a biosensor array distinct location.

### Blocking of Non-Specific Binding on a Biosensor Surface

Sugars, such as trehalose and dextran, can be used to reduce non-specific interactions between specific binding substances immobilized on a biosensor surface and other small molecules and proteins in the solution. Sugars useful in this invention include, e.g., monosaccharides, disaccharides, trisaccharides, tetrasaccharides, pentasaccharides, and other polysaccharides and oligosaccharides, such as dextran. Sugars, which are non-proteinaceous substances, can be used to block, or reduce non-specific binding of molecules to, among other things, protein-coated biosensor surfaces, such a BIND™ sensor plates. The sugar does not interfere with specific protein-ligand interactions (such as streptavidin-biotin interactions). As used herein, “oligosaccharide” refers to a sugar that had between around three and ten monosaccharide units, and “polysaccharide” refers to a sugar that has more than around 10 monosaccharide units, but can have more than three thousand monosaccharide units. Dextran is an example of a complex, branched polysaccharide made of many glucose molecules joined into chains of varying lengths. Sugars can be used to block, for example, amine-functionalized surfaces, aldehyde-functionalized surfaces, and carboxyl-functionalized surfaces. Reduction in non-specific binding can be measured by, for example, the methodologies described in Examples 3 and 4.

These sugar blockers, such as disaccharide blockers, can also be used as a storage solution to ship protein-coated plates. For example, after a desired protein is immobilized on an aldehyde functionalized biosensor surface, such as streptavidin on a sensor plate, a solution of trehalose is added the plate before packaging. Trehalose binds to the aldehyde surface and blocks any remaining aldehyde groups. When the recipient receives the biosensor, the surface is pre-blocked with the disaccharide molecules. The sugars offer an inert way to keep the plate wet or stable without allowing chemical reactions that might otherwise make the plate un-useful. In this embodiment, the sugars act as temporary protecting groups. The sugars are thought to form hemi-acetals with the aldehydes resulting in short-lived “covalent” bonds that may be easily removed/reversed through simple re-dox chemistry. If a protein is added to a surface before the sugars are added, the sugars serve to stabilize the protein by keeping a layer of important structure maintaining water molecules in close proximity with the immobilized protein. The sugars do not interfere with specific protein-ligand interactions (such as streptavidin-biotin interactions) due to their molecular size and uniformly high hydrophobicity. The sugars also serve the function of keeping the protein from becoming denatured. The addition of the sugar allows the surfaces with immobilized proteins to be shipped in a more stable and possibly semi-dry state.

Sugars that are useful in this invention include reducing and non-reducing sugars. Reducing sugars contain an aldehyde group, and include members such as fructose, glucose, glyceraldehyde, lactose, and maltose. Because reducing sugars can react with amines, amine functionalized surfaces can be blocked with reducing sugars, for example glyceraldehydes. Monosaccharides can also attach to amine-functionalized surfaces. Aldehyde functionalized surfaces can be blocked with sugars containing aldehyde groups that also contain hydroxyl groups. Both reducing and non-reducing sugars contain hydroxyl groups that react with aldehyde groups on an aldehyde functionalized surface of the sensor, thereby blocking the surface. Trehalose and sucrose fall within the category of non-reducing sugar because they do not contain an aldehyde group.

The aldehyde functionalized surfaces can be used for protein attachment. Sugars can react with the aldehydes and block them, thereby preventing proteins from reacting with the aldehyde groups on the surface that are not bound to a specific binding substance. Surfaces of the present invention may also contain some amine groups. For example, a high density amine surface that is converted to an aldehyde-containing surface may not have all of its amine groups converted and therefore remain on the surface. Therefore, both reducing and non-reducing sugars can be used to block amine and aldehyde groups on the surfaces, such that protein added as an analyte during an assay will bind only to the immobilized proteins, but not to the other parts of the surface that contain amine and aldehyde groups.

It is believed that trehalose, and other disaccharide molecules, attach to aldehyde functional groups that are left unreacted after a target protein is immobilized on the surface, possibly via a hemi-acetal. The bond is reversible with simple re-dox chemistry and can be made stable for the time scale of a typical assay of only about a few hours. Therefore, trehalose seems to bind to aldehyde surfaces in a stable fashion.

One embodiment of the invention provides a biosensor comprising a plurality of specific binding substances bound to surface-attached aldehyde groups and a plurality of sugar molecules bound to surface-attached aldehyde groups. That is, the biosensor was aldehyde-functionalized and sugar and specific binding substances were added to the biosensor so that the specific binding substances and sugars bound to the biosensor surface-attached aldehyde groups.

Another embodiment of the invention provides a package containing a biosensor with specific binding substances bound to surface-attached aldehyde groups and a storage solution comprising sugar molecules. The surface-attached aldehyde groups are amine groups that were added to the surface to make it an aldehyde functionalized surface.

The invention illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms, without changing their ordinary meanings. The terms and expressions that have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

The following are provided for exemplification purposes only and are not intended to limit the scope of the invention described in broad terms above. All references cited in this disclosure are incorporated herein by reference.

### EXAMPLE 1

Fabrication of a SWS Biosensor

The detailed manufacture process of an SWS biosensor has been described previously. See, e.g., Cunningham et al., Sensor and Actuators B 6779, 1-6 (2002), incorporated herein by reference. Specifically, an optical-grade polymer film was used as a support of SWS sensor. A UV-curable acrylic-based polymer coating was coated onto the film and replicated using a silicon mask that has 96 circles corresponding to the standard format of a 96-well micro-titer plate, which circles form an SWS structure. A UV lamp RC600, provided by Xenon Corporation (Woburn, Mass.), was used to cure the coating after the replication. Subsequently, a titanium dioxide layer and a silicone dioxide layer were deposited onto the top of the surface.

### Immobilization of One or More Specific Binding Substances

The following protocol was used on a calorimetric resonant reflective biosensor to functionalize the surface with amine functional groups. Amine groups can be used as a general-purpose surface for subsequent covalent binding of several types of linker molecules.

A glass version of a biosensor is cleaned by immersing it into piranha etch (70/30% (v/v) concentrated sulfuric acid/30% hydrogen peroxide) for 12 hours. The biosensor was washed thoroughly with water. The biosensor was dipped in 3% 3-aminopropyl-triethoxysilane solution in dry acetone for 1 minute and then rinsed with dry acetone and air-dried. The biosensor was then washed with water.

A semi-quantitative method is used to verify the presence of amino groups on the biosensor surface. One biosensor from each batch of amino-functionalized biosensors is washed briefly with 5 mL of 50 mM sodium bicarbonate, pH 8.5. The biosensor is then dipped in 5 mL of 50 mM sodium bicarbonate, pH 8.5 containing 0.1 mM sulfo-succinimidyl-4-O-(4,4′-dimethoxytrityl)-butyrate (s-SDTB, Pierce, Rockford, Ill.) and shaken vigorously for 30 minutes. The s-SDTB solution is prepared by dissolving 3.0 mg of s-SDTB in 1 mL of DMF and diluting to 50 mL with 50 mM sodium bicarbonate, pH 8.5. After a 30 minute incubation, the biosensor is washed three times with 20 mL of ddH₂O and subsequently treated with 5 mL 30% perchloric acid. The development of an orange-colored solution indicates that the biosensor has been successfully functionalized with amines; no color change is observed for untreated glass biosensors.

The absorbance at 495 nm of the solution after perchloric acid treatment following the above procedure can be used as an indicator of the quantity of amine groups on the surface. In one set of experiment, the absorbance was 0.627, 0.647, and 0.728 for Sigma slides, Cel-Associate slides, and in-house biosensor slides, respectively. This indicates that the level of NH₂ functionalization of the biosensor surface is comparable in the functionalized commercially available microarray glass slides.

After following the above protocol for functionalizing the biosensor with amine, a linker molecule can be attached to the biosensor. When selecting a cross-linking reagent, issues such as selectivity of the reactive groups, spacer arm length, solubility, and cleavability should be considered. The linker molecule, in turn, binds the specific binding substance that is used for specific recognition of a binding partner. As an example, the protocol below has been used to bind a biotin linker molecule to the amine-functionalized biosensor.

Protocol for Functionalizing Amine-Coated Biosensor with Biotin

Wash an amine-coated biosensor with PBS (pH 8.0) three times. Prepare sulfo-succinimidyl-6-(biotinamido)hexanoate (sulfo-NHS-LC-biotin, Pierce, Rockford, Ill.) solution in PBS buffer (pH 8) at 0.5 mg/ml concentration. Add 2 ml of the sulfo-NHS-LC-biotin solution to each amine-coated biosensor and incubate at room temperature for 30 min. Wash the biosensor three times with PBS (pH 8.0). The sulfo-NHS-LC-biotin linker has a molecular weight of 556.58 and a length of 22.4 Å. The resulting biosensors can be used for capturing avidin or streptavidin molecules.

Protocol for Functionalizing Amine-Coated Biosensor with Aldehyde

Prepare 2.5% glutaraldehyde solution in 0.1 M sodium phosphate, 0.1% sodium cyanoborohydride, pH 7.0. Add 2 ml of the glutaraldehyde solution to each amine-coated biosensor and incubate at room temperature for 30 min. Wash the biosensor three times with PBS (pH 7.0). The glutaraldehyde linker has a molecular weight of 100.11. The resulting biosensors can be used for binding proteins and other amine-containing molecules. The reaction proceeds through the formation of Schiff bases, and subsequent reductive amination yields stable secondary amine linkages. In one experiment, where a coated aldehyde slide was compared to a commercially available aldehyde slide (Cel-Associate), ten times higher binding of streptavidin and anti-rabbit IgG on the slide made by the above method was observed.

Protocol for Functionalizing Amine-coated Biosensor with NHS

25 mM N,N′-disuccinimidyl carbonate (DSC, Sigma Chemical Company, St. Louis, Mo.) in sodium carbonate buffer (pH 8.5) was prepared. 2 ml of the DSC solution was added to each amine-coated biosensor and incubated at room temperature for 2 hours. The biosensors were washed three times with PBS (pH 8.5). A DSC linker has a molecular weight of 256.17. Resulting biosensors are used for binding to hydroxyl- or amine-containing molecules. This linker is one of the smallest homobifunctional NHS ester cross-linking reagents available.

In addition to the protocols defined above, many additional surface functionalization and molecular linker techniques have been reported that optimize assay performance for different types of biomolecules. Most common of these are amine surfaces, aldehyde surfaces, and nickel surfaces. The functionalized surfaces, in turn, can be used to attach several different types of chemical linkers to the biosensor surface, as shown in Table 2. While the amine surface is used to attach several types of linker molecules, the aldehyde surface is used to bind proteins directly, without an additional linker. A nickel surface is used exclusively to bind molecules that have an incorporated histidine (“his”) tag. Detection of “his-tagged” molecules with a nickel activated surface is well known (Sigal et al. (1996) Anal Chem., vol. 68, p. 490).

Table 1 demonstrates an example of the sequence of steps that are used to prepare and use a biosensor, and various options that are available for surface functionalized chemistry, chemical linker molecules, specific binding substances and binding partners molecules. Opportunities also exist for enhancing detected signal through amplification with larger molecules such as HRP or streptavidin and the use of polymer materials such as dextran or TSPS to increase surface area available for molecular binding.

TABLE 1

| | Surface | | | | Label |
|---|---|---|---|---|---|
| Bare | Acti- | Linker | Receptor | Detected | Molecule |
| Sensor | vation | Molecule | Molecule | Material | (Optional) |
| Glass | Amino | SMPT | Sm | Peptide | Enhance |
| | | | m'cules | | sensi- |
| | | | | | tivity |
| Polymers | | NHS-Biotin | Peptide | Med Protein | 1000x |
| optional to | Alde- | DMP | Med | Lrg Protein | HRP |
| enhance | hyde | | Protein | IgG | |
| sensitivity | Ni | NNDC | Lrg | Phage | Strepta- |
| 2-5x | | | Protein | | vidin |
| | | | IgG | | |
| Dextran | | His-tag | | Cell | |
| TSPS | | Others . . . | cDNA | cDNA | |

### EXAMPLE 2

### Aldehyde Surface Functionalization and Testing

The following example provides chemical functional groups on the surface of a calorimetric resonant biosensor for binding proteins, peptides, nucleic acids, cells, small molecules, small organic molecules, other chemical and/or biological molecules, and the like. Such chemical functional groups are of interest in proteomic, genomic, pharmaceutical, drug discovery, diagnostics, environmental, chemical and similar study areas and/or industries. This example addresses the development of a coating process that provides a high density of functional aldehyde binding sites using chemical reagents that do not alter or degrade biosensor structures. Another issue this example addresses is the development of test methods to verify the presence of aldehyde binding sites on the functionalized surface of calorimetric resonant biosensors.

Prior sensor surfaces have been coated with surface amine groups. A simple method for coating a sensor surface with amine is to directly expose the cleaned surface to polylysine. An example is a glass slide surface used for microarray printing. An alternative to coat a surface with amine is to covalently attach amine-coating molecules to the surface, such as attaching silanes on glass or thiols on gold, both of which are well known. Some aldehyde modified slides are also commercially available (e.g., CEL Associates and NoAb BioDiscoveries, infra) for printing arrays.

This example describes a process that provides a high density of functional aldehyde binding sites on the surface of a calorimetric resonant biosensor without altering or degrading the biosensor structure.

In order to verify the presence of aldehyde groups, current calorimetric and fluorescent methods typically employ samples in solution, wherein detection can be more sensitive than samples on a dry surface. Performing surface characterization of chemical groups less than about 50 Angstroms thick on a dry and uneven (i.e., not entirely flat) surface has also proven to be a difficult task.

A calorimetric resonant biosensor surface can be functionalized with amine groups, wherein the surface can be a biosensor comprising a plastic, glass, epoxy, or polymer substrate as described herein. The amine-functionalized surface can be rinsed with coupling buffer. A coupling buffer can be formulated in phosphate buffered saline (PBS) at a pH of about 7.4. An aldehyde solution comprising cyanoborohydride can be added to the biosensor surface. The aldehyde solution can comprise about 10% glutaraldehyde containing about 100 mM cyanoborohydride. The surface can be allowed to stand, for example, for about 4 hours, at approximately room temperature. The biosensor surface can be washed with, for example, coupling buffer.

In order to test for the presence of aldehyde groups on the surface of a biosensor, many methods may be used, including, but not limited to, radioactive, calorimetric, fluorescence, X-ray photoelectron spectroscopy (XPS), Fourier transform infrared spectroscopy (FTIR), atomic force microscopy (AFM), and the like.

A fluorescence test for aldehyde-surface functionalization can comprise, for example, cutting aldehyde-treated and non-treated biosensors into about 2×2 cm² pieces. The aldehyde-coated surface can be exposed to a fluorescent dye, which is capable of being excited with visible light. For example, a fluorescent hydrazine derivative like ALEXA 647-hydrazine (Molecular Probes, Portland, Oreg.) in PBS (pH 7.4) solution, which can be prepared with a final concentration of the dye at about 100 μg/mL is used. About 100 μL of the dye solution can be dispensed on a piece of coverslip and the biosensor piece placed face down on the dye solution. The biosensor can be incubated with the dye for about 1 hour at about room temperature. The coverslip and dye solution can be discarded and the biosensor surface can be rinsed, for example, 3× in distilled, deionized water. The biosensor pieces can be placed in a petri dish with, for example, 20 mL of distilled, deionized water and washed for about 1 hour on a rocking platform. The biosensor pieces can be dried with N₂. Water droplets can be used to hold down biosensor pieces onto a glass slide for scanning using, for example, an Affymetrix® 428™ scanner. Fluorescence is read for each biosensor piece such that the amount of aldehyde binding sites is determined.

A colorimetric resonant biosensor comprises, for example, a high refractive index material deposited on a grating that comprises a low refractive index material as described herein. The high refractive index material can be, for example, zinc sulfide, titanium dioxide, indium tin oxide, tantalum oxide, or silicon nitride, while the low refractive index material can be, for example, glass, plastic, polymer, or epoxy. In one embodiment, the high refractive index material of the biosensor is optionally coated with SiO₂. The aldehyde-coating on the surface of a colorimetric biosensor used can be any thickness, including less than about 50 Angstroms thick. Furthermore, the biosensor surface can be uneven (i.e., not flat). The biosensor surface can be dry prior to the detection of aldehyde surface activation.

Using the aldehyde functionalization protocol and the fluorescence testing method above, the aldehyde-coated dyed biosensor typically shows at least a ten fold excess in fluorescence, relative to a non-aldehyde surface functionalized biosensor. Table 2 shows a comparison of plastic biosensors with no aldehyde functionalization (blank) and aldehyde-functionalized biosensors, all subjected to the fluorescence test procedure. The protocol and method above showed that the surface density of the coated aldehyde was higher than that obtained by methods used by commercial vendors (e.g., CEL Associates, Pearland, Tex. and NoAb BioDiscoveries, (Mississauga, ON, Canada). Table 3 shows a comparison of glass slides with no aldehyde functionalization (blank, shown average of 4 samples) and aldehyde-functionalized slides, obtained by methods of the invention and commercial vendors (by Cel-Associate and by NoAb), all subjected to the fluorescence test procedure.

TABLE 2

| | Fluorescence |
|---|---|
| Plastic Biosensor Sample | Intensity (counts) |
| Blank 1 - no aldehyde | 785 |
| Blank 2 - no aldehyde | 746 |
| Sample 1 - aldehyde coated by NoAb | 6431 |
| Sample 2 - aldehyde coated by NoAb | 5070 |
| Sample 3 - aldehyde coated using methods | 17280 |
| of the invention | |
| Sample 4 - aldehyde coated using methods | 14109 |
| of the invention | |

TABLE 3

| | Fluorescence |
|---|---|
| Glass Slide Sample | Intensity (counts) |
| Blank 1 - no aldehyde | 825 ± 85 |
| Sample group 1 - aldehyde coated by Cel-Assoc | 7684 ± 796 |
| Sample group 2 - aldehyde coated by NoAb | 15847 ± 2020 |
| Sample group 3 - aldehyde coated using | 35486 ± 7664 |
| methods of the invention | |

Aldehyde surface functionalization can also be tested using a protein-binding test. For example, an approximately 100 μg/mL solution of protein A in PBS can be prepared at a pH of approximately 7.4 and added to aldehyde-coated and untreated biosensor pieces and incubated for about 1 hour at about room temperature. The biosensor can be washed with PBS solution comprising 1% (w/v) BSA about 3 times. The biosensor pieces can be placed in the wash solution to wash for about 15 minutes at about room temperature. The biosensor pieces can be rinsed with PBS buffer at about pH 7.4 at about room temperature. A labeled solution of a suitable antibody is added to the surface of the biosensors and incubated. For example, a solution of about 20 μg/mL rabbit anti-goat IgG-ALEXA 647 (Molecular Probes) can be used and the biosensor can be incubated in the dark for approximately 30 minutes. The biosensor can be washed with PBS buffer comprising about 0.05% Tween™ 20 at about pH 7.4 (PBST solution) about 3 times. The biosensor can be washed with deionized water and dried with N₂. Detection can comprise, for example, scanning the biosensor with an Affymetrix® 428™ scanner to obtain fluorescence reading.

Using an aldehyde coating process and protein-binding test method, it has been shown that aldehyde-coated biosensors bind protein A/IgG about 5× better than un-coated biosensors that have been subjected to the same protein A/IgG treatment (See Table 4).

TABLE 4

| | Fluorescence |
|---|---|
| Plastic Biosensor Sample | Intensity (counts) |
| Blank 1 - no aldehyde | 4249 |
| Blank 2 - no aldehyde | 3525 |
| Blank 3 - no aldehyde | 4572 |
| Blank 4 - no aldehyde | 3976 |
| Sample 1 - aldehyde coated | 18759 |
| Sample 2 - aldehyde coated | 22212 |
| Sample 3 - aldehyde coated | 15170 |
| Sample 4 - aldehyde coated | 19525 |

Table 5 shows how an aldehyde functionalized, plastic-based, colorimetric resonant biosensor array responded when it was sequentially exposed to PBS, BSA, and anti-BSA. Six biosensors were monitored simultaneously. At step 1, all biosensors were exposed to PBS (pH 7.4) solution to obtain the baseline for the biosensors. Then at step 2, sensors 3-6 were exposed to 0.5 mg/mL of BSA (made in PBS) while biosensors 1 and 2 (used as reference) were exposed to only PBS. An average signal of approximately 0.38 nm was observed on the BSA-bound biosensors while the reference biosensors remained at baseline. Finally, at step 3, biosensors 3 and 4 were exposed to 0.5 mg/mL of anti-BSA while biosensors 1 and 2 were again exposed to only PBS. It was found that the binding of anti-BSA on BSA (biosensors 3 and 4) gave an additional ˜1.4 nm signal above baseline. As controls, at this step, biosensors 5 and 6 were exposed to PBS and 0.5 mg/mL of BSA, respectively, where their responses remained at the step 2 level.

TABLE 5

| | | Step 1 | Step 2 | Step 3 |
|---|---|---|---|---|
| | | Signal | Signal | Signal |
| | | Wavelength | Wavelength | Wavelength |
| | Expose to | Shift | Shift | Shift |
| | solution | (nm) | (nm) | (nm) |
| Sensor 1 - | Step 1 PBS + | 0.000 | −0.009 | −0.020 |
| Reference | Step 2 PBS + | | | |
| | Step 3 PBS | | | |
| Sensor 2 - | Step 1 PBS + | 0.000 | 0.009 | 0.020 |
| Reference | Step 2 PBS + | | | |
| | Step 3 PBS | | | |
| Sensor 3 - | Step 1 PBS + | −0.002 | 0.384 | 1.473 |
| Sample | Step 2 BSA + | | | |
| | Step 3 anti-BSA | | | |
| Sensor 4 - | Step 1 PBS + | −0.002 | 0.350 | 1.425 |
| Sample | Step 2 BSA + | | | |
| | Step 3 anti-BSA | | | |
| Sensor 5 - | Step 1 PBS + | 0.000 | 0.389 | 0.398 |
| Control | Step 2 BSA + | | | |
| | Step 3 PBS | | | |
| Sensor 6 - | Step 1 PBS + | −0.002 | 0.380 | 0.390 |
| Control | Step 2 BSA + | | | |
| | Step 3 BSA | | | |

The processes, methods, and results described herein are generally applicable due to the wide range of detection applications that can be performed using an aldehyde coated surface, including, but not limited to, binding of proteins, peptides, nucleic acids, cells, small molecules, small organic molecules, other chemical and/or biological molecules, and the like. Such applications are of interest in many fields, including, but not limited to, proteomic, genomic, pharmaceutical, drug discovery, diagnostics, environmental, chemical, and the like.

### EXAMPLE 3

### Blocking of Streptavidin (SA) Surface with 5% Trehalose

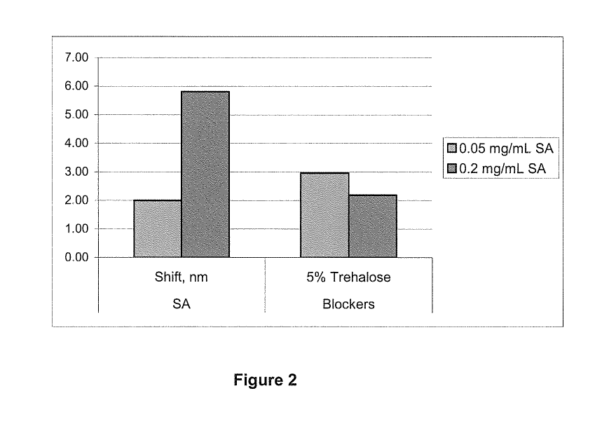

40 μl of 5% Trehalose solution was added to SA immobilized wells (0.05 mg/ml SA and 0.2 mg/ml SA) and control wells (Trehalose alone) and were allowed to bind for 2 hrs. The plates were washed and the peak wavelength value (PWV) shift was measured (See Table 6 and FIG. 2). The SA shift was the PWV of SA binding to the aldehyde surface, while the blocker shift was the PWV shift obtained after the blocker was allowed to bind to the unreacted aldehyde functional groups on the SA-immobilized aldehyde surface. The unreacted aldehydes are more easily accessed by the small blocker molecule than the bigger SA molecules. The blocker shift is lower with higher SA concentration likely because there are less aldehydes available for binding the blocker in these wells.

TABLE 6

| SA concentration | SA shift | Blocker shift |
|---|---|---|
| during immobilization | Nm | 5% Trehalose |
| 0.05 mg/mL SA | 2.00 | 2.96 |
| 0.2 mg/mL SA | 5.81 | 2.19 |
| Aldehyde surface (no | — | 2.91 |
| streptavidin) | | |

### EXAMPLE 4

### Trehalose Efficacy to Reduce Non-Specific Binding

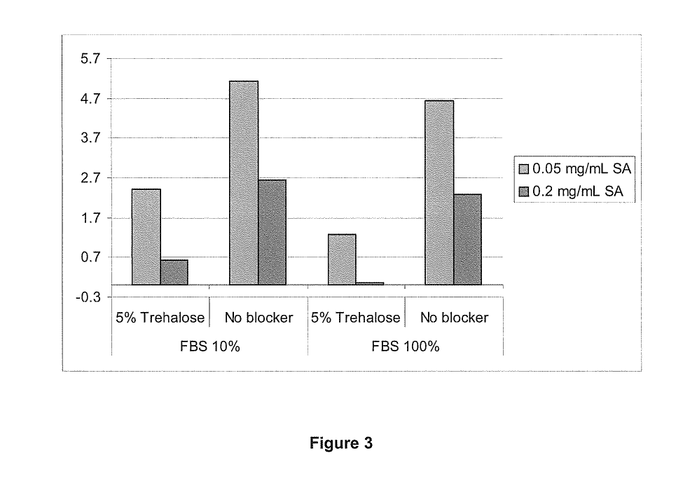

In order to test the efficacy of trehalose to reduce non-specific binding, the ability of trehalose to reduce Fetal Bovine Serum (FBS) binding to the SA surface was measured. 10% and 100% commercially available FBS was allowed to react with either the SA surface with the 2 nm PWV shift or the 5.8 nm PWV shift, and the resulting shift measured (see Table 7 and FIG. 3). The shifts were normalized to the trehalose shift by considering the resulting signal after the surface has been blocked to be zero, or baseline. The change in binding due to the protein present in FBS is then measured and reported as non-specific binding.

TABLE 7

| | |
|---|---|
| FBS 10% binding to | FBS 100% binding to |

| SA shift, | 5% | No | 5% | No |
|---|---|---|---|---|
| nm | Trehalose | Trehalose | Trehalose | Trehalose |
| 2 | 2.41 | 5.13 | 1.27 | 4.65 |
| 5.8 | 0.62 | 2.64 | 0.07 | 2.28 |

These results indicate that FBS is binding to unreacted aldehydes on the surface. Further, these results demonstrate that even at low immobilization density of SA, which have more unreacted aldehyde groups still available on the surface, blocking with trehalose reduced non-specific binding.

Trehalose reduced the non-specific binding of proteins from FBS to the SA surface. The highest shift reduction was seen with the 5.8 nm SA surface indicating that the optimum choice of the blocker is dependant on the surface density of the immobilized target protein. In other words, for low surface densities, where the surface is sparsely covered, either a small molecule blocker or a large molecule blocker, like an inert protein, could be used. However, at high surface densities, when there is high protein coverage of the surface, the blocker should be small enough to penetrate the protein layer and bind to the unreacted aldehydes.

Another consideration for choice of blocker is the intended application of the surface. Surfaces that are used for applications such as protein-protein interactions, for example, the screening of antibodies, generally have low densities of target proteins, while high-immobilization density surfaces are used in, for example, the detection of small molecule binding, such as in drug screening.

### EXAMPLE 5

### Trehalose Affect on Specific Protein-Protein Interactions

In order to test the affect of trehalose on specific protein-protein interactions, the ability of trehalose to interfere with the ability of biotin to bind to a SA surface was measured. Biotin in either 10% or 100% FBS was allowed to react with the SA surface with the 5.8 nm PWV shift, and the resulting shift measured (see Table 8). The theoretical shift resulting from Biotin binding, shown in parenthesis, was calculated from the following formula: (Mol. Wt. of Biotin/Mol. Wt. of SA)×# of binding sites on SA for biotin×SA shift, or (244/55000)×4 (binding sites)×5.8 nm (SA shift). A clear biotin binding signal was seen in all cases. Therefore, trehalose does not seem to interfere with biotin binding as was seen with other blockers.

TABLE 8

| | FBS 10% | | FBS 100% | |
|---|---|---|---|---|
| SA | 5% | No | 5% | No |
| Shift | Trehalose | Trehalose | Trehalose | Trehalose |
| 5.8 nm | 0.09 (0.104) | 0.08 (0.104) | 0.1 (0.104) | 0.12 (0.104) |

### EXAMPLE 6

### Trehalose Affect on HSA-Warfarin Protein-Small Molecule System

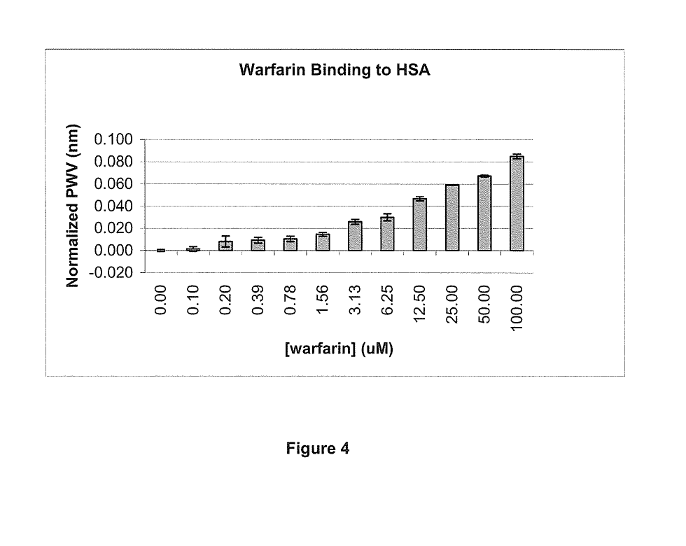

Trehalose can be used to reduce the non-specific binding on other protein-small molecule systems. For example, trehalose can block the non-specific binding associated with the interaction of Human Serum Albumin (HSA) and Warfarin. The assay was performed in 1% DMSO. The HSA was blocked with 5% trehalose. The results are shown in Table 8 and FIG. 4. Trehalose blocking of non-specific interactions of warfarin to the surface enabled detection of specific binding to HSA below 1 μM. It is difficult to detect binding at such low concentrations is trehalose if not added.

TABLE 9

| Warfarin | | |
|---|---|---|
| concentration, | PWV shift of Warfarin | Std. |
| μM | binding to HAS, nm | Dev. |
| 0.00 | 0.000 | 0.001 |
| 0.10 | 0.001 | 0.002 |
| 0.20 | 0.008 | 0.005 |
| 0.39 | 0.009 | 0.003 |
| 0.78 | 0.011 | 0.002 |
| 1.56 | 0.015 | 0.002 |
| 3.13 | 0.026 | 0.002 |
| 6.25 | 0.030 | 0.003 |
| 12.50 | 0.047 | 0.002 |
| 25.00 | 0.059 | 0.000 |
| 50.00 | 0.067 | 0.001 |
| 100.00 | 0.085 | 0.002 |

### EXAMPLE 7

### Trehalose Affect on Carbonic Anhydrase (CA)-Carboxysulfonamide (CBS) Protein-Small Molecule System

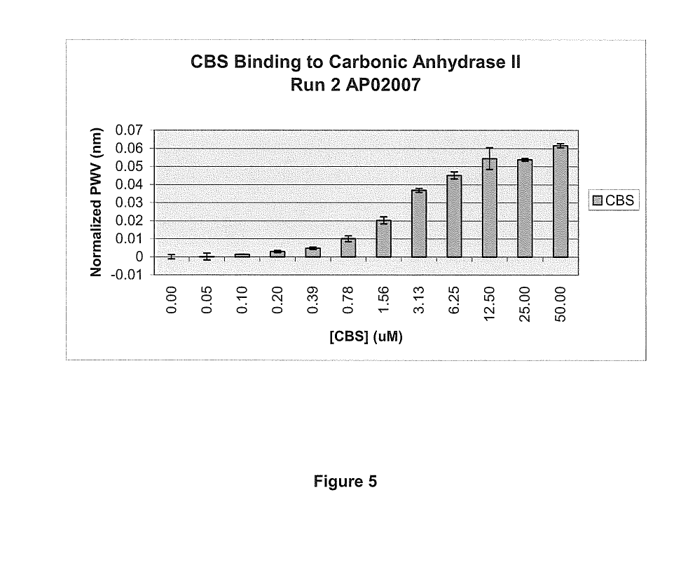

Trehalose can be used to reduce the non-specific binding on other protein-small molecule systems. For example, trehalose can block the non-specific binding associated with the interaction of CBS and Carbonic Anhydrase. The CA surface was blocked with 5% trehalose. The results are shown in Table 9 and FIG. 5. Trehalose blocking of non-specific interactions of CBS to the surface enabled detection of specific binding to CA below 1 μM. It is difficult to detect binding at such low concentrations if trehalose if not added.

TABLE 9

| | PWV shift of | |
|---|---|---|
| CBS | CBS binding to | |
| concentration, μM | CA, nm | Std. Dev. |
| 0.00 | 0.000 | 0.001 |
| 0.05 | 0.000 | 0.002 |
| 0.10 | 0.001 | 0.000 |
| 0.20 | 0.003 | 0.001 |
| 0.39 | 0.005 | 0.001 |
| 0.78 | 0.010 | 0.002 |
| 1.56 | 0.020 | 0.002 |
| 3.13 | 0.037 | 0.001 |
| 6.25 | 0.045 | 0.002 |
| 12.50 | 0.054 | 0.006 |
| 25.00 | 0.054 | 0.001 |
| 50.00 | 0.062 | 0.001 |

### EXAMPLE 8

### Blocking Non-Specific Protein Binding on a Biosensor Surface

Disaccharide molecules, such as trehalose, can be used to block non-specific binding of proteins or other molecules to, for example, a protein coated biosensor surface, such as a BIND™ sensor plate.

Disaccharide molecules can also be used as a storage solution to ship protein-coated plates. A desired protein is immobilized on a biosensor surface, such as streptavidin on the BIND™ sensor plate. A solution of disaccharide molecules, such as trehalose, is added to the wells in the plate and packaged. Thus, when the user receives the biosensor, the surface is preblocked with the disaccharide molecules.

### EXAMPLE 9

### Sugars Binding to Amine Surfaces

Lactose and glyceraldehyde solutions in water at 5% and 2.5% respectively were added to a BIND™ sensor plate functionalized with amine functional groups for 2 hrs. The plate was then washed thoroughly with water and stored overnight in water. The plates were then washed again with water and the binding of sugars was measured. The following table shows the difference between shifts before overnight incubation and post-incubation. Note that the shifts have not changed significantly overnight, indicating a stable bond between the sugar blockers and amine functional groups on the surface.

| | | PWV change after |
|---|---|---|
| Sugar solution | PWV change after | over night |
| binding to amine | 2 hr incubation and | incubation and |
| surface | wash, nm | wash, nm |
| Lactose 5% solution | 4.86 | 4.82 |
| Glyceraldehydes | 1.75 | 1.72 |
| 2.5% solution | | |

We claim:

1. A method for reducing non-specific binding on a surface, wherein the surface is aldehyde-functionalized, amine-functionalized, or a combination thereof, comprising:
treating the surface with sugar molecules, whereby non-specific binding on the surface is reduced.

2. The method of claim 1, wherein the sugar molecules comprise disaccharides.

3. The method of claim 2, wherein the disaccharides are trehalose molecules or comprise trehalose molecules.

4. The method of claim 1, wherein the sugar molecules are dextran sulfate or comprise dextran sulfate.

5. The method of claim 1, wherein the surface is a biosensor surface.

6. The method of claim 5, wherein the biosensor is a calorimetric resonant reflectance biosensor.

7. The method of claim 1, wherein the sugar molecules comprise monosaccharides, disaccharides, polysaccharides, trisaccharides, tetrasaccharides, pentasaccharides, or a combination thereof.

8. The method of claim 1, wherein the surface is an amine-functionalized surface.

9. The method of claim 8, wherein the sugar molecules comprises lactose, glyceraldehydes, or a combination thereof.

10. The method of claim 1, wherein the sugar molecules comprise trehalose and lactose.

11. The method of claim 1, wherein the sugar molecules comprise trehalose and glyceraldehyde.

12. A biosensor comprising a plurality of specific binding substances bound to surface-attached aldehyde groups and a plurality of sugar molecules bound to surface-attached aldehyde groups.

13. The biosensor of claim 12, wherein the sugar molecules comprise disaccharides.

14. The biosensor of claim 13, wherein the disaccharides are trehalose molecules.

15. The biosensor of claim 15, wherein the sugar molecules are disaccharides.

16. The biosensor of claim 13, wherein the disaccharides are trehalose molecules.

17. The biosensor of claim 12, wherein the sugar molecules comprise dextran.

18. The biosensor of claim 12, wherein the sugar molecules are dextran.

19. The biosensor of claim 12, wherein the specific binding substances are proteins.

20. The biosensor of claim 19, wherein the proteins comprise streptavidin.

21. A package containing a biosensor with specific binding substances bound to surface-attached aldehyde groups and a storage solution comprising sugar molecules.

22. The package of claim 21, wherein the sugar molecules comprise disaccharides.

23. The package of claim 22, wherein the disaccharides comprise trehalose.

24. The package of claim 21, wherein the sugar molecules comprise dextran.

25. The package of claim 21, wherein the sugar molecules are dextran.
